(19) United States
(12) Patent Application Publication
(10) Pub. No.: US 2012/0029561 A1
(43) Pub. Date: Feb. 2, 2012

(54) Coated Colored Medical Devices

(75) Inventor: James R. Olson, Norwich, CT (US)

(73) Assignee: TELEFLEX MEDICAL INCORPORATED, Research Triangle Park, NC (US)

(21) Appl. No.: 13/193,062

(22) Filed: Jul. 28, 2011

Related U.S. Application Data
(60) Provisional application No. 61/368,457, filed on Jul. 28, 2010.

(51) Int. Cl. A61B 17/04; B05D 7/00

(52) U.S. Cl. 606/228; 427/2.31

(57) ABSTRACT

The present invention provides, in some embodiments, a medical securement device such as sutures, yarns, meshes, filaments, tapes, knits, and composite braids made from fiber materials like UHMWPE which are coated in whole or in part with a colored coating solution. This invention provides surgeons with improved recognition of suture ends in surgery by construction of sutures with ends having different colors. Additionally, the invention provides surgeons with a means for color coding or marking of medical devices for proper recognition of various devices used in surgery for particular tasks and surgical procedures.

### CROSS REFERENCE TO RELATED APPLICATION

This application claims priority to earlier filed U.S. Provisional Patent Application No. 61/368,457, filed on Jul. 28, 2010, the disclosure of which is hereby incorporated by reference, in its entirety.

### FIELD OF THE INVENTION

The present invention generally relates to medical devices for holding together tissue. More particularly, the present invention pertains to a coated colored construction for medical devices.

### BACKGROUND OF THE INVENTION

Sutures, yarns, filaments, meshes, tapes, knits, and composite braids are commonly used to hold together tissue that has been severed by injury, incision, or surgery to aid in the healing process or to apply pressure to blood vessels to stop bleeding. These medical devices can be absorbable or non-absorbable. Non-absorbable tissue joining devices are made from inherently colorless materials such as silk, polypropylene, polyester, nylon, or ultra-high molecular weight polyethylene (UHMWPE).

Sutures are commonly in either braided multifilament or monofilament construction, but are sometimes also known in a twisted construction. The form of a suture is usually dictated by the stiffness or fiber modulus of its constituent fiber. Sutures require good pliability for their handling and knotting properties. Thus, fibers that are too stiff cannot be used in a monofilament form for larger suture sizes but rather in bundles of small denier filaments. These strands of multifilament are then braided together to form a suture. As the suture diameter gets larger, a core is usually added inside the braided sheath. Fibers with a modulus below about 600,000 psi can be used in monofilament form, although lower modulii are preferred. Thus, polyester and UHMWPE sutures are used in multifilament braids, while polypropylene is used as a monofilament suture. Some materials like nylon 6, 6 with borderline properties are made in both multifilament and monofilament constructions. Natural fibers like silk that are fine denier can only be used in multifilament constructions.

Synthetic and most natural materials used to make sutures and other surgical devices are without color. With the two basic constructions of braided multifilament and monofilament many types of sutures would be indistinguishable from other sutures even though their properties might be quite different. Additionally, it can be advantageous to color code devices with different properties or purposes. Thus, the value of coloring in medical devices has long been recognized. Not only does color provide distinction between different types of sutures and other medical devices but is also known to aid the surgeon to keep track of sutures in the blood field during surgery.

Sutures and other implantable devices can only contain specific colorants listed in the Code of Federal Regulations (CFR) for a particular suture material to be used. The regulations also put a limit on the maximum weight percent for each colorant for each suture material. Thus, there are limitations in colors available for all suture materials. There are also certain inherent limitations to how color can be added to some suture materials based on their polymer properties and how they are processed into sutures.

For example, UHMWPE high strength yarn, which can be used to make sutures, yarns, meshes, filaments, tapes, knits, and composite braids, is highly oriented and has a high percentage of crystallinity, which makes it unsuitable for normal solvent dyeing processes. Additionally, colorants can only be added during fiber production, and only one colorant is FDA approved for use in sutures made from polyethylene.

The advent of endoscopic surgery procedures has put additional pressure on surgeons to correctly identify sutures and their respective ends for proper tying of knots in confined spaces. During suturing it may be necessary for a physician to distinguish between the ends of similar sutures, which becomes more difficult when both ends of the sutures have an identical appearance of either no color or the same color.

Thus, it is helpful to the surgeon to have choices in the coloring of sutures. One method of adding color choices to a material that has few color options, like braids of UHMWPE, is to co-braid one or more strands of another suture material that contains an approved color. The colored strands of suture can be multifilament or monofilament. However, these braid changes can invariably cause changes in the performance properties of the overall braid. A method of coloration that preserves the performance properties of the braid is most desired.

Another way to aid a surgeon in distinguishing an incoming end from an outgoing end of a suture, is to use a “half and half” suture, in which one half or one end is colored and the other half or end remains white. For example, Teleflex Medical of 1295 Main Street, Coventry, Conn. 06238, has made a polyester braided tape since 1998 in which one half of it has been dyed with D&C Green No. 6. So the tape has one end that is green and one end that is undyed white. Although the half and half does allow the surgeon to distinguish between the incoming and outgoing ends of one individual suture strand, it offers no help when two or more of the same type of suture is being used. In these cases, there is no differentiation between the various incoming and outgoing ends of suture from each other.

Additionally, more variety in the color of surgical sutures, yarns, meshes, filaments, tapes, knits, and composite braids made from UHMWPE would allow for color coding or marking of devices for particular tasks in surgical procedures, which would be important for proper recognition of various devices.

Therefore, it would be advantageous to provide sutures, yarns, meshes, filaments, tapes, knits, composite braids or other surgical securement devices made from non-colored material like UHMWPE a method to incorporate different colors beyond the uncolored (white) and limited colorants that are currently approved for use.

### SUMMARY OF THE INVENTION

The foregoing needs are met, to a great extent, by the present invention, wherein in some embodiments a suture or medical device that is capable of overcoming the disadvantages described herein at least to some extent is provided.

In accordance with an embodiment of the invention a suture includes an elongate woven braid of fibers. The braid can be made from one fiber material like UHMWPE or other nonabsorbable and absorbable materials. In this invention, color is added to the uncolored suture by coating with a colorant dissolved in a polymer solution. The color coating can be applied to all or parts of the suture.

The colored coating solution can also be applied to cobraided sutures. For example, the elongate woven braid can include one or more first ends comprising ultra-high molecular weight polyethylene having a first portion and a second portion. The elongate woven braid can also include at least one second end formed from a fiber suitable for forming into a braided suture. The second portion is coated with a solution comprising a polymer, a solvent, and a colorant such that the second portion of the suture takes on the color of the colorant.

In accordance with another aspect of the present invention the suture can include an overcoat, and the overcoat can be in the form of a high molecular weight co-polymer. The polymer can also comprise a low molecular weight co-polymer. The suture can include a bioactive agent, such as an anti-microbial, anti-thrombogenic, tissue healing/growth or cell attachment agent. Additionally, the polymer can contain anti-microbial agent triclosan and glyceryl monostearate. The suture can also have a reduced weight percent of colorant for a larger suture size.

In accordance with another embodiment of the present invention, a method of coating fibers used to create a fully or partially colored medical device includes creating a solution including a colorant and polymer in a solvent or co-solvents. The amount of colorant and polymer in the solution can be adjusted to meet visibility and contrast requirements for the suture or medical device. The solution containing the colorant can be applied to the fiber. Additionally, the suture can be passed through a dryer to remove excess solvent and/or harden the coating to form a skin on the suture. The color coated suture can also be washed to remove any residual coating on the surface. The color coated suture can also be coated again with a clear coating of polymer.

In accordance with another aspect of the present invention, the method can include applying an overcoat to the suture, the overcoat can be made from a high molecular weight co-polymer. The method can also include adding anti-microbial agent triclosan and glyceryl monostearate to the solution.

In accordance with still another aspect of the present invention, a method of coating fibers used to create a medical device can include creating a solution containing a polymer, a solvent, and a colorant. The method can also include applying the solution containing the colorant to the fibers. Additionally, the method can include passing the fiber through a dryer to remove excess solvent, and adjusting the percentage of colorant in the solution based on suture size.

In accordance with yet another aspect of the present invention, the method can include reducing the percentage of colorant in the solution for larger sutures. The method can also include applying an overcoat of an high molecular weight co-polymer. Additionally, the method can include adding anti-microbial agents triclosan and glyceryl monostearate to the solution.

As such, those skilled in the art will appreciate that the conception upon which this disclosure is based may readily be utilized as a basis for the designing of other structures, methods and systems for carrying out the several purposes of the present invention. It is important, therefore, that the claims be regarded as including such equivalent constructions insofar as they do not depart from the spirit and scope of the present invention.

### BRIEF DESCRIPTION OF THE DRAWINGS

FIGS. 1A, 1B, and 1C illustrate a non-colored braided suture that can be transformed into either a fully, half, or partially colored suture in accordance with an embodiment of the invention.

FIG. 2 illustrates a cross section of a color coated suture in accordance with an embodiment of the invention where the color resides in the coating.

FIG. 3 illustrates a co-braided half color coated suture in accordance with an embodiment of the invention.

FIG. 4 illustrates a graph of the correlation between percentage by weight of dye and absorption of the dye.

### DETAILED DESCRIPTION

The present invention provides, in some embodiments, a medical securement device such as sutures, yarns, meshes, filaments, tapes, knits, and composite braids made from UHMWPE which are coated in whole or in part with a colored coating solution. This invention provides surgeons with improved recognition of suture ends in surgery by construction of sutures with ends having different colors. Additionally, the invention provides surgeons with a means for color coding or marking of medical devices for proper recognition of various devices used in surgery for particular tasks and surgical procedures.

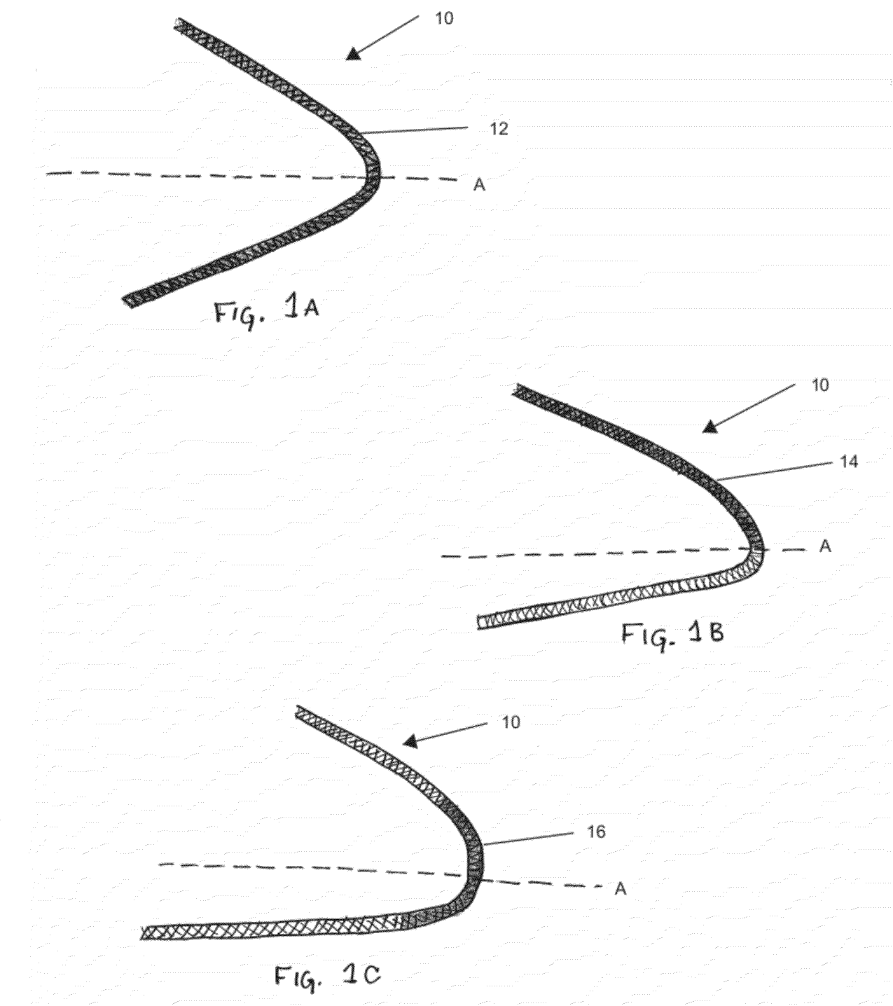

The invention will now be described with reference to the drawing figures, in which like reference numerals refer to like parts throughout. FIG. 1A illustrates a braided suture 10 preferably made from UHMWPE. Alternately, the suture can be formed from any suitable fiber for braiding a surgical suture. A colorant can be added to a solution of a polymer in a solvent which can then be directly applied to the surface of the medical device. As illustrated in FIG. 1A, the suture 10 is braided and can be completely coated with color 12 by dipping it into a solution containing a colorant. FIG. 1B illustrates the braided suture 10 made from UHMWPE, or any other suitable fiber for braiding a surgical suture. The suture illustrated in FIG. 1B can be half coated by dipping it into a solution containing a colorant. The portion of the suture 14 above axis A is shown as being dipped into the solution containing the colorant. FIG. 1C illustrates the braided suture 10 made from UHMWPE, or any other suitable fiber for braiding a surgical suture. The suture illustrated in FIG. 1C can be partially coated by dipping it into a solution containing a colorant. As shown the portion of the suture 16 surrounding axis A is dipped into the solution containing the colorant. Alternately, different portions of the suture can be coated with the solution containing the colorant and the suture shown in FIGS. 1A, 1B, and 1C and these figures should not be considered limiting. The solution can be made from various components and applied to the sutures in various ways which will be described in more detail below.

For example, the sutures shown in FIGS. 1A, 1B, and 1C can be made from UHMWPE and can be passed in a normal spool to spool coating process through a solution of polycaprolactone or polycaprolactone-co-polyglycolic acid copolymer in methylene chloride with added concentration of a colorant like D&C Violet #2, D&C Green #6 or D&C Blue #6. D&C Violet #2 is listed in 21CFR74.3602 for use in several absorbable materials, including polycaprolactone and polycaprolactone-co-polyglycolic acid for use in sutures.

The coating material is preferably polycaprolactone based, that is a homopolymer or a copolymer of ε-caprolactone with another absorbable monomer like glycolide, lactide, trimethylene carbonate or p-dioxanone. In general, any material can be used that can contain a colorant and provide good coating to a medical device, suture or fiber construction could be used in this invention. Thus, nonabsorbable as well as absorbable polymers or copolymers can be considered for coatings.

For example, a size 2 suture made from sixteen ends of white UHMWPE yarn with a 16 carrier braider is passed in a normal spool to spool coating process through a solution of polycaprolactone or polycaprolactone-co-polyglycolic acid copolymer in methylene chloride with added concentration of a colorant like D&C Violet #2, D&C Green #6 or D&C Blue #6. This polymer will adhere to the surface of the suture and also carry the colorant with it leaving a highly colored device. This process can also be used to overcoat and color all or portions of devices that already have color by normal methods of coloring.

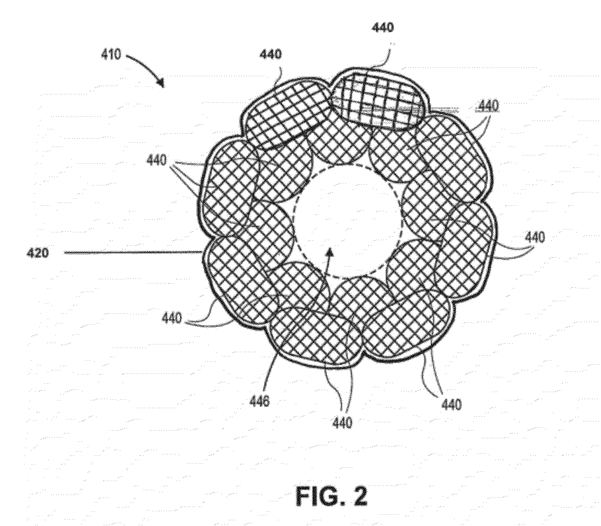

FIG. 2 illustrates a cross section of a color coated suture 410 in accordance with an embodiment of the invention where the color resides in the coating. The suture 410 includes monofilament or multifilament ends 440 made from UHMWPE. The ends 440 made from UHMWPE are generally colorless. The suture 410 as shown in FIG. 2 has been dipped into a solution carrying a colorant creating a coating 420 on the outside of the suture 410. FIG. 2 illustrates that the colorant resides in the coating on the suture and is of sufficient concentration to impart color to the suture surface.

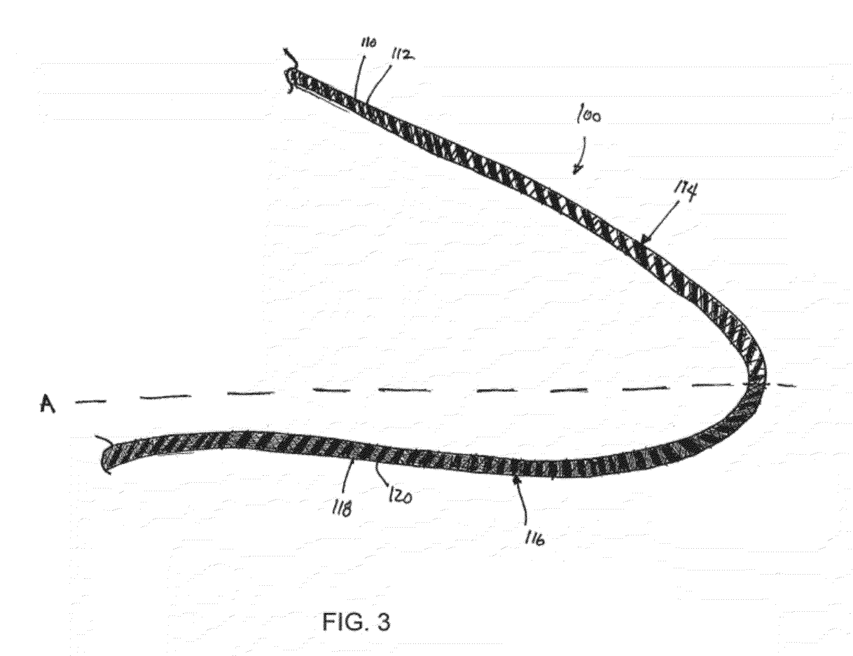

As shown in FIG. 3, another example, a co-braid of size 2 suture 100 can be made from braiding eight ends of generally colorless UHMWPE yarn with eight ends of green polyester yarn, creating a pattern of colorless UHMWPE 110 and green polyester yarn 112. The half coated suture 100, shown in FIG. 3 has a first half 114, above axis A, which remains a green-white co-braid and second half 116, below axis A, which is coated with the solution with the colorant, creating a suture that is half green-white co-braid and half colored. Thus, this suture construction can be dip coated into a solution of D&C Violet #2 and polycaprolactone-co-polyglycolic acid to color over portions of the green-white co-braid to give completely violet portions useful for coding sutures for endoscopic procedures. As shown in FIG. 3, a half 114 of the suture 100 above axis A remains uncoated with the colorless UHMWPE 110 and the green polyester yarn 112 creating a striped pattern. The half 116 of the suture below axis A is coated in the manner described above to provide a violet region 118 and a region colored green and also coated with violet 120.

Coating solutions can consist of one or more solvents, a colorant and a polymer. The polymer should have good coating properties for the suture or device being coated. The solution can include solvents in which the polymer and colorant will have good solubility. However, the solvent preferably will not dissolve the device to be coated. Preferably, polycaprolactone homopolymer and polycaprolactone copolymers can be used as coatings in this invention. Other aliphatic polyesters and their copolymers like polydioxanone, polylactides, and polyhydroxyacids can also be used. Solvents that can be used with polycaprolactone or its copolymers include methylene chloride, ethyl acetate, acetone, and xylene. Copolymers of caprolactone with glycolide, para-dioxanone, lactide, or trimethylene carbonate can also be used as coatings in this invention.

The sutures shown in FIGS. 1A, 1B, 1C, 2 and 3 can be manufactured in a variety of different ways with a variety of different solutions. Some examples of these combinations are detailed below. However, these examples should not be considered limiting, as there are numerous other ways to coat the sutures. As one example, a coating solution is prepared by dissolving 6.0 grams of polycaprolactone-co-glycolide in 84.4 g methylene chloride. 9.4 g of ethanol and 0.2 g of D&C Violet #2 is added to the coating solution. This color coating solution contains 6% (w/w) of PCL-PGA and 0.2% (w/w) of the colorant D&C Violet #2. This coating solution can be used to color coat a UHMWPE suture or fiber. The coating process can be carried out “spool to spool.” In one example of the “spool to spool” process, the uncoated suture on a spool is pulled off and passed through the coating solution by three inch diameter godet rolls rotating at 11 RPM. The suture is then taken up on another spool equipped with traverse and tension controlled speed takeup. After passing through the coating solution, but before reaching the pulling godets, the suture line can travel through a drying area to remove excess solvent. The coated suture has a light violet color that appears homogeneous on the suture surface.

A 36 inch sample of the coated violet suture described above is weighed, extracted with methylene chloride, dried and weighed again, such that the percent coating on the suture is found to be 2.1% (w/w). Analysis of the extracted coating shows a colorant content of 2.47% in the coating. This calculates to 0.10% concentration of the D&C Violet #2 in the overall color coated suture. In other additional examples, coating solutions are made to contain 0.4% and 0.6% D&C Violet #2, 0.4% D&C Blue #6, and 0.4% D&C Green #6. The coating solutions are used to coat the following suture types, such as one example “Force Fiber” sutures made of braided UHMWPE made by Teleflex Medical Incorporated:

Size 2 white UHMWPE Force Fiber

Size 0 white UHMWPE Force Fiber

Size 2-0 white UHMWPE Force Fiber

Size 2 white Cottony polyester

Size 2 white co-braid UHMWPE and white polyester (50:50)

Size 2 blue co-braid. 14 ends UHMWPE and 2 ends blue polypropylene

Size 3-0 blue co-braid. UHMWPE and blue polypropylene

Size 2 black co-braid, 14 ends UHMWPE, 1 end clear nylon, 1 end black nylon

Size 3-0 black co-braid, UHMWPE and black nylon

Size 2 green polyblend, 8 ends UHMWPE and 8 ends green polyester PGA mesh

In another example, a coating solution are prepared using acetone as the sole solvent instead of methylene chloride/ethanol co-solvent to give a 0.4% solution of D&C Violet #2. This coating solution can be used to coat a size 2 poly-L-lactic acid suture braid as described above. Acetone can be used because the PLLA is soluble in methylene chloride. This coating solution can also be used to dip coat PLLA mesh.

The coating with colorant can be applied to both braids of 100% UHMWPE fibers and to co-braids and polyblends with other fiber materials like PET polyester, PLLA, nylon and polypropylene. Coating solutions that contain 0.2% by weight D&C Violet #2 and 6% by weight polycaprolactone copolymer can yield coatings on sutures that contain 4.7% and 0.1% dye in the coating and overall suture, respectively. It should be noted that these examples are not limiting and higher or lower concentrations of colorant in the coating solutions can be used to yield higher or lower concentrations in the coating on the device.

In another example a coating solution containing 0.4% D&C Violet #2 in 6% PCL-PGA (90:10) is prepared and used to coat a size 2 white UHMWPE suture as described in Example 1. The coated suture was tested for percent weight coating and concentration of the dye in the coating and in then the concentration of the dye in the suture as a whole was calculated. These results are presented in Table 1.

In another example, a coating solution containing 0.6% D&C Violet #2 in 2% PCL-PGA (90:10) can be prepared and used to coat a size 2 white UHMWPE suture as described above. The coated suture was tested for percent weight coating and concentration of the dye in the coating and then the concentration of the dye in the suture as a whole was calculated. These results are presented in Table 1.

In another example, a coating solution containing 0.2% D&C Violet #2 in 4% PCL-PGA (90:10) is prepared and used to coat a size 2 white UHMWPE suture as described above. The coated suture was tested for percent weight coating and concentration of the dye in the coating and then the concentration of the dye in the suture as a whole was calculated. These results are presented in Table 1.

TABLE 1

| COATING | COATING | SUTURE | SUTURE | SUTURE |
|---|---|---|---|---|
| SOLUTION | SOLUTION % | COATING | COATING % | % D&C |
| % PCL- | D&C VIOLET | % | D&C | VIOLET |
| PGA | #2 | WEIGHT | VIOLET#2 | #2 |
| 6 | 0.2 | 2.10 | 4.73 | 0.10 |
| 6 | 0.4 | 2.47 | 7.50 | 0.18 |
| 6 | 0.6 | 3.40 | 7.80 | 0.26 |
| 2 | 0.2 | 0.90 | 14.8 | 0.13 |
| 4 | 0.2 | 1.48 | 7.79 | 0.11 |

In another example, a coating solution containing 0.4% D&C Green #6 in 6% PCL-PGA (90:10) is prepared and used to coat size 2 white UHMWPE Force Fiber as described above. The coated suture can have 3.65 percent weight coating and concentration of the dye in the coating of 4.78%. The concentration of the dye in the suture as a whole can be approximately 0.17%.

In another example, a coating solution containing 0.4% D&C Blue #6 in 6% PCL-PGA (90:10) is prepared and used to coat size 2 white UHMWPE Force Fiber as described above. The coated suture can have 2.72 percent weight coating and a light blue color.

In another example, a coating solution containing 0.1% (w/w) D&C Violet #2 in 6% PCL-PGA (90:10) is prepared and used to coat a size 2 white UHMWPE suture as described above. After drying the color coated suture is washed in ethanol to remove any colorant at the outside surface of the coating. The washed color coated suture is then overcoated with a 6% PCL-PGA solution in ethyl acetate. The second polymer coating contains no colorant and serves to seal the colorant in the inner coating. The weight average molecular weights of the polymer coatings do not need to be the same. In fact, it is advantageous to use a lower molecular weight (LMW) polymer for the inner coating layer and a higher molecular weight polymer (HMW) polymer for the overcoat or top coat layer. The ratio of LMW/HMW in overall coating can affect handling properties of suture, hence particular ratios are evaluated for acceptability. The coating processes described here give acceptable handling suture properties, although other combinations may be useful.

The coated UHMWPE coated sutures of the examples above can have homogeneous coatings that was well adhered to the braided surfaces. Additional examples of different suture materials and surgical devices coated with different solutions appear in Table 2, below. It is also important to note that these examples are not limiting and other concentrations of coating and/or dyes can be performed to give optimum desired coloring in the sutures.

TABLE 2
MC = methylene chloride; ETOH = ethanol; ETOAC = ethyl acetate

| SUTURE | SUTURE | COATING | |
|---|---|---|---|
| MATERIAL | SIZE | PROCESS | COLORANT |
| WHITE UHMWPE | 0 | 6% PCL-PGA, | 0.4% D&C |
| | | MC/ETOH | VIOLET #2 |
| WHITE UHMWPE | 2-0 | 6% PCL-PGA, | 0.4% D&C |
| | | MC/ETOH | VIOLET #2 |
| WHITE | 2 | 6% PCL-PGA, | 0.4% D&C |
| UHMWPE/WHITE | | MC/ETOH | VIOLET #2 |
| PET | | | |
| WHITE | 2 | 6% PCL-PGA, | 0.4% D&C |
| UHMWPE/GREEN | | MC/ETOH | VIOLET #2 |
| PET | | | |
| WHITE | 3-0 | 6% PCL-PGA, | 0.4% D&C |
| UHMWPE/BLUE | | MC/ETOH | VIOLET #2 |
| POLYPROPYLENE | | | |
| WHITE | 2 | 6% PCL-PGA, | 0.4% D&C |
| UHMWPE/BLUE | | MC/ETOH | VIOLET #2 |
| POLYPROPYLENE | | | |
| WHITE | 3-0 | 6% PCL-PGA, | 0.4% D&C |
| UHMWPE/BLACK | | MC/ETOH | VIOLET #2 |
| NYLON | | | |
| WHITE | 2 | 6% PCL-PGA, | 0.4% D&C |
| UHMWPE/BLACK | | MC/ETOH | VIOLET #2 |
| NYLON | | | |
| WHITE PET | 2 | 6% PCL-PGA, | 0.4% D&C |
| | | MC/ETOH | VIOLET #2 |
| WHITE UHMWPE | 5 | 6% PCL-PGA, | 0.4% D&C |
| | | MC/ETOH | VIOLET #2 |
| WHITE UHMWPE | 7-0 | 6% PCL-PGA, | 0.4% D&C |
| | | MC/ETOH | VIOLET #2 |
| WHITE PLLA | 2 | 6% PCL-PGA, | 0.4% D&C |
| | | ACETONE | VIOLET #2 |
| BEIGE PGA | 2-0 | 6% PCL-PGA, | 0.4% D&C |
| | | MC/ETOH | VIOLET #2 |
| UHMWPE | 2 | 6% PCL, | 0.4% D&C |
| | | MC/ETOH | VIOLET #2 |
| UHMWPE | 2 | 6% PCL-PGA, | 0.1% D&C |
| | | MC/ETOH; | VIOLET #2 |
| | | ETOH WASH | |
| UHMWPE | 2 | 6% PCL-PGA, | 0.1% D&C |
| | | MC/ETOH; | VIOLET #2 |
| | | ETOH WASH; | |
| | | 6% PCL- | |
| | | PGA/ETOAC | |
| UHMWPE | 2 | 6% PCL-GMS, | 0.4% D&C |
| | | MC/ETOH | VIOLET #2, |
| | | | 1% |
| | | | TRICLOSAN |
| UHMWPE | 2-0 | 6% PCL-PGA, | 0.4% D&C |
| | | MC/ETOH | VIOLET #2, |
| | | | 1% |
| | | | TRICLOSAN |
| WHITE PET | 2 | 6% PCL-PGA, | 0.4% D&C |
| | | MC/ETOH | GREEN #6 |
| WHITE PET | 2 | 6% PCL-PGA, | 0.4% D&C |
| | | MC/ETOH | BLUE #6 |
| WHITE UHMWPE | 2 | 6% PCL-PGA, | 0.4% D&C |
| | | MC/ETOH | GREEN #6 |
| WHITE UHMWPE | 2 | 6% PCL-PGA, | 0.4% D&C |
| | | MC/ETOH | BLUE #6 |
| WHITE | 2 | 6% PCL-PGA, | 0.4% D&C |
| UHMWPE/WHITE | | MC/ETOH | BLUE #6 |
| PET | | | |
| WHITE | 2 | 6% PCL-PGA, | 0.4% D&C |
| UHMWPE/WHITE | | MC/ETOH | GREEN #6 |
| PET | | | |
| WHITE | 2 | 6% PCL-PGA, | 0.4% D&C |
| UHMWPE/BLACK | | MC/ETOH | BLUE #6 |
| NYLON | | | |
| WHITE | 2 | 6% PCL-PGA, | 0.4% D&C |
| UHMWPE/BLACK | | MC/ETOH | GREEN #6 |
| NYLON | | | |
| WHITE PLLA | MESH | 6% PCL-PGA, | 0.4% D&C |
| | | ACETONE | VIOLET #2 |
| BEIGE PGA | MESH | 6% PCL-PGA, | 0.4% D&C |
| | | MC/ETOH | VIOLET #2 |

Other solvents like acetone can be used instead of methylene chloride/ethanol co-solvent, in cases where the material to be coated, such as PLLA, is soluble in methylene chloride. In general, other possible solvents include those in which polycaprolactone is soluble, like acetone, ethyl acetate, toluene and xylene.

Additionally, colored coating can also be used on already colored yarns or monofilaments in braided sutures. This coloring method can be used in coloring selected sections of sutures for enhanced identification in surgical procedures. This method can also be used in preparing half and half sutures, and continuous half and half sutures. Additionally, this method can be used for marking suture areas for bone anchor placement in endoscopic and other surgeries.

The colored coating solution can be used to provide color to surgical mesh made from poly-L-lactic acid (PLLA) or polyglycolic acid (PGA). The meshes can be dipped into the respective coating solutions and allowed to air dry. This method of applying color can be used for placing color on select areas of a mesh in addition to the whole mesh. Additionally adding color to one side of the mesh may be possible by application of the coating solution to one side of the mesh or a medical device.

The coating solution can also be used to add one or more useful ingredients to the coating on the suture. Useful ingredients include bioactives, tissue healing/growth, anti-microbial, anti-thrombogenic and controlled release agents. For example, an antimicrobial agent triclosan and glyceryl monostearate (GMS) are added to provide a suture with anti-bacterial activity. The GMS can be used to control the rate of release from the polycaprolactone homopolymer or copolymer coating material. The additional agents or ingredients can be used with either PCL or PCL-PGA coating material.

The examples in Tables 1 and 2 show colorants that can be applied to both braids of 100% UHMWPE fibers and to co-braids and polyblends with other fiber materials like PET polyester, PLLA, nylon and polypropylene. The colored coating can be applied to many other suture materials alone like polyester and nylon nonabsorbable sutures and polylactic acid (PLA) and polyglycolic acid (PGA) absorbable sutures. Other nonabsorbable and absorbable sutures, in either braid or monofilament form can also be color coated.

The amount of color in the coating and in the suture as a whole can be altered by changing the relative concentrations of the colorant and coating material in the coating solution. Higher or lower concentrations of colorant in the coating solutions can yield higher or lower concentrations in the coating on the device. Also, the amount of colorant on the surface of the coated sutures can be altered through heating the coated surface, washing the coated surface or coating over the coating surface with either the same coating material or a new coating material. The overcoating material can be either nonabsorbable or absorbable. Possible overcoating materials can include all of the first coating materials described like polycaprolactone and its copolymers with other monomers. In addition other polymers can be used like polyethylene glycols, polyethylene oxides, polypropylene oxides, also in random or block copolymers.

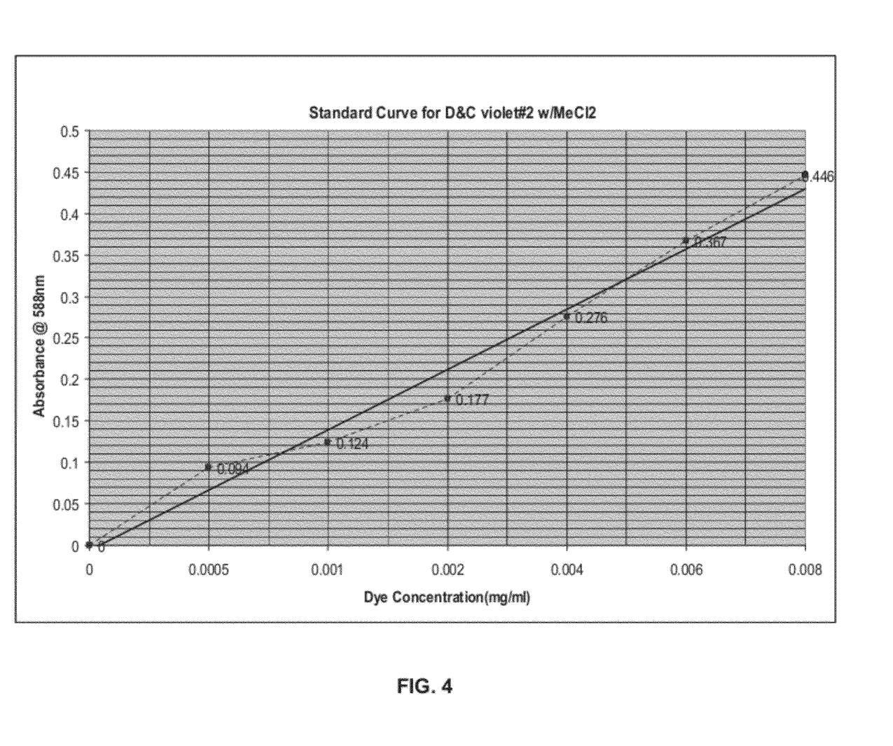

It is possible that larger suture sizes will appear in a darker violet shade than smaller suture sizes when coated with the same concentration of D&C violet #2 in the coating solution. However, constant shades can be obtained by reducing the weight percentage of the dye in the coating solution for the larger suture size, as shown in Tables 3 and 4, below. For example, a size 5 UHMWPE braided suture was found to have the same shade of violet when coated with a 6% LMW-PCL-PGA/0.0375% D&C Violet #2 as a size 2 UHMWPE braided suture with a 6% LMW-PCL/PGA 0.050% D&C Violet #2 coating solution. These sutures can then be overcoated with a 1.5% HMW PCL/PGA coating solution. FIG. 4 also illustrates the correlation between dye concentration and the absorbance of the suture for D&C Violet #2 with MeCl₂.

TABLE 3
Dye wt % in coated UHMWPE braided suture

| | | | | Dye | | | | |
|---|---|---|---|---|---|---|---|---|
| | | | | concentration | | | | |
| Sample | | | Absorbance | per std. curve | Initial | Pickup | % Dye in | % Dye in |
| ID | Size | Coating Process | @ 588 nm | below (mg/ml) | Wt (mg) | Wt (mg) | coating | suture |
| 40-30-1 | 5 | 6% LMW | 0.083 | 0.00060 | 413.6000 | 18.3 | 0.16393 | 0.00725 |
| | | PCL:PGA/0.05% | | | | | | |
| | | D&C | | | | | | |
| | | Violet2 + EtOH | | | | | | |
| | | wash + clear coat | | | | | | |
| | | w/1.5% HMW | | | | | | |
| | | PCL:PGA | | | | | | |
| 40-30-2 | 5 | 6% LMW | 0.084 | 0.00060 | 413.5000 | 18.1 | 0.16575 | 0.00726 |
| | | PCL:PGA/0.05% | | | | | | |
| | | D&C | | | | | | |
| | | Violet2 + EtOH | | | | | | |
| | | wash + clear coat | | | | | | |
| | | w/1.5% HMW | | | | | | |
| | | PCL:PGA | | | | | | |
| | | | | | | | AVG = | 0.00725 |
| 40-30-3 | 2 | 6% LMW | 0.041 | 0.00032 | 205.7000 | 8 | 0.20000 | 0.00778 |
| | | PCL:PGA/0.05% | | | | | | |
| | | D&C | | | | | | |
| | | Violet2 + EtOH | | | | | | |
| | | wash + clear coat | | | | | | |
| | | w/1.5% HMW | | | | | | |
| | | PCL:PGA | | | | | | |
| 40-30-4 | 2 | 6% LMW | 0.043 | 0.00033 | 206.0000 | 8.2 | 0.20122 | 0.00801 |
| | | PCL:PGA/0.05% | | | | | | |
| | | D&C | | | | | | |
| | | Violet2 + EtOH | | | | | | |
| | | wash + clear coat | | | | | | |
| | | w/1.5% HMW | | | | | | |
| | | PCL:PGA | | | | | | |
| | | | | | | | AVG = | 0.00789 |
| 40-30-5 | 2_0 | 6% LMW | 0.016 | 0.000124 | 69.7000 | 2.7 | 0.22963 | 0.00890 |
| | | PCL:PGA/0.05% | | | | | | |
| | | D&C | | | | | | |
| | | Violet2 + EtOH | | | | | | |
| | | wash + clear coat | | | | | | |
| | | w/1.5% HMW | | | | | | |
| | | PCL:PGA | | | | | | |
| 40-30-6 | 2_0 | 6% LMW | 0.016 | 0.000124 | 68.1000 | 2.4 | 0.25833 | 0.00910 |
| | | PCL:PGA/0.05% | | | | | | |
| | | D&C | | | | | | |
| | | Violet2 + EtOH | | | | | | |
| | | wash + clear coat | | | | | | |
| | | w/1.5% HMW | | | | | | |
| | | PCL:PGA | | | | | | |
| | | | | | | | AVG = | 0.00900 |
| 40-30-7 | 5_0 | 6% LMW | 0.012 | 0.000096 | 15.9000 | 0.5 | 0.96000 | 0.03019 |
| | | PCL:PGA/0.05% | | | | | | |
| | | D&C | | | | | | |
| | | Violet2 + EtOH | | | | | | |
| | | wash + clear coat | | | | | | |
| | | w/1.5% HMW | | | | | | |
| | | PCL:PGA | | | | | | |
| 40-30-8 | 5_0 | 6% LMW | 0.013 | 0.000097 | 16.5000 | 0.6 | 0.80833 | 0.02939 |
| | | PCL:PGA/0.05% | | | | | | |
| | | D&C | | | | | | |
| | | Violet2 + EtOH | | | | | | |
| | | wash + clear coat | | | | | | |
| | | w/1.5% HMW | | | | | | |
| | | PCL:PGA | | | | | | |
| | | | | | | | AVG = | 0.02979 |

TABLE 4
Coating Wt % Pickup for Violet Coated UHMWPE Braided Suture

| | | | | | | % |
|---|---|---|---|---|---|---|
| Sample | | | Initial | Final | Pickup | coat- |
| ID | Size | Process | wt (g) | wt (g) | Wt (g) | ing |
| 40-30-1 | 5 | 6% LMW | 0.4136 | 0.3953 | 0.0183 | 4.42 |
| | | PCL:PGA/0.05% | | | | |
| | | D&C | | | | |
| | | Violet2 + EtOH | | | | |
| | | wash + clear coat | | | | |
| | | w/1.5% HMW | | | | |
| | | PCL:PGA | | | | |
| 40-30-2 | 5 | 6% LMW | 0.4135 | 0.3954 | 0.0181 | 4.38 |
| | | PCL:PGA/0.05% | | | | |
| | | D&C | | | | |
| | | Violet2 + EtOH | | | | |
| | | wash + clear coat | | | | |
| | | w/1.5% HMW | | | | |
| | | PCL:PGA | | | | |
| | | | | | AVG = | 4.40 |
| 40-30-3 | 2 | 6% LMW | 0.2057 | 0.1977 | 0.0080 | 3.89 |
| | | PCL:PGA/0.05% | | | | |
| | | D&C | | | | |
| | | Violet2 + EtOH | | | | |
| | | wash + clear coat | | | | |
| | | w/1.5% HMW | | | | |
| | | PCL:PGA | | | | |
| 40-30-4 | 2 | 6% LMW | 0.2060 | 0.1978 | 0.0082 | 3.98 |
| | | PCL:PGA/0.05% | | | | |
| | | D&C | | | | |
| | | Violet2 + EtOH | | | | |
| | | wash + clear coat | | | | |
| | | w/1.5% HMW | | | | |
| | | PCL:PGA | | | | |
| | | | | | AVG = | 3.93 |
| 40-30-5 | 2_0 | 6% LMW | 0.0697 | 0.0670 | 0.0027 | 3.87 |
| | | PCL:PGA/0.05% | | | | |
| | | D&C | | | | |
| | | Violet2 + EtOH | | | | |
| | | wash + clear coat | | | | |
| | | w/1.5% HMW | | | | |
| | | PCL:PGA | | | | |
| 40-30-6 | 2_0 | 6% LMW | 0.0681 | 0.0657 | 0.0024 | 3.52 |
| | | PCL:PGA/0.05% | | | | |
| | | D&C | | | | |
| | | Violet2 + EtOH | | | | |
| | | wash + clear coat | | | | |
| | | w/1.5% HMW | | | | |
| | | PCL:PGA | | | | |
| | | | | | AVG = | 3.70 |
| 40-30-7 | 5_0 | 6% LMW | 0.0159 | 0.0154 | 0.00050 | 3.14 |
| | | PCL:PGA/0.05% | | | | |
| | | D&C | | | | |
| | | Violet2 + EtOH | | | | |
| | | wash + clear coat | | | | |
| | | w/1.5% HMW | | | | |
| | | PCL:PGA | | | | |
| 40-30-8 | 5_0 | 6% LMW | 0.0165 | 0.0159 | 0.00060 | 3.64 |
| | | PCL:PGA/0.05% | | | | |
| | | D&C | | | | |
| | | Violet2 + EtOH | | | | |
| | | wash + clear coat | | | | |
| | | w/1.5% HMW | | | | |
| | | PCL:PGA | | | | |
| | | | | | AVG = | 3.39 |

Since the invention is directed at improvements in the cover or outer sheath of the suture, this invention is applicable to both coreless sutures and sutures with cores. The cores can be of any construction including twisted or non-twisted multifilament, braided cores or monofilament cores. However, for most sizes of UHMWPE braided suture a coreless construction is preferred. This is because a coreless construction gives better knotting properties for sutures made from high modulus UHMWPE yarn. For other suture materials a core is normally needed to prevent the sheath from collapsing in suture sizes greater than about USP size 5-0 to a flat profile which is not optimum for suturing. The extremely high fiber modulus of UHMWPE of over 75 GPa is probably the main factor that keeps it from flattening during its handling, as opposed to the fiber modulus of about 3-10 GPa in other synthetic braided fibers. The coreless UHMWPE braid will flatten when it is compressed by the forces of knotting. This phenomenon contributes to its lower knot profile and improved knotting properties.

The coating process can also be carried out by a number of different process methods. One of the preferred methods is to wind the suture on a frame and then dip half of it into the solution. Of course, the frames can be of a variety of sizes and configurations including flat, square, rectangular or round. For some sutures the coating process could also take place on other devices or even in a skein form. The coating process can also be carried out on pre-cut pieces of suture that are then half coated in bundles of the sutures. Certainly other aids to enhance the coating process can be envisioned. For example, it has also been discovered that the junction between the half dyed end and the other end can also be improved by applying anti-wicking compounds like silicone fluids to the junction line before the coating process.

The many features and advantages of the invention are apparent from the detailed specification, and thus, it is intended by the appended claims to cover all such features and advantages of the invention which fall within the true spirit and scope of the invention. Further, because numerous modifications and variations will readily occur to those skilled in the art, it is not desired to limit the invention to the exact construction and operation illustrated and described, and accordingly, all suitable modifications and equivalents may be resorted to falling within the scope of the invention.

What is claimed is:

1. A suture comprising:
an elongate woven braid of fibers comprising:
one or more first ends;
one or more second ends;
wherein the one or more first ends and one or more second ends together define a first portion and a second portion of the suture;
wherein the second portion is coated with a solution comprising a polymer, a solvent, and a colorant such that the second portion of the suture takes on the color of the colorant and the first portion retains a color of the original fiber.

2. The suture of claim 1, wherein the one or more first ends comprise an ultra-high molecular weight polyethylene.

3. The suture of claim 1, wherein the one or more second ends comprise nylon.

4. The suture of claim 1, wherein the first ends and the second ends comprise an ultra-high molecular weight polyethylene.

5. The suture of claim 1, wherein the coating polymer is polycaprolactone based.

6. The suture of claim 5, wherein the polycaprolactone comprises one of a homopolymer of ε-caprolactone or a copolymer of ε-caprolactone.

7. The suture of claim 6, wherein the polycaprolactone further comprises one of glycolide, lactide, trimethylene carbonate, or p-dioxanone.

8. The suture of claim 6, further comprising an overcoat.

9. The suture of claim 8, wherein the overcoat comprises a high molecular weight co-polymer.

10. The suture of claim 9, wherein the polymer comprises a low molecular weight co-polymer.

11. The suture of claim 6, further comprising a bioactive agent.

12. The suture of claim 11, further comprising an anti-microbial, anti-thrombogenic, tissue healing/growth or cell attachment agent.

13. The suture of claim 12, further comprising anti-microbial agent triclosan, and glyceryl monostearate.

14. The suture of claim 6, further comprising a reduced weight percent of colorant for a larger suture size.

15. A method of coating fibers used to create a medical device comprising:
creating a solution comprising:
a polymer;
a solvent;
adding a colorant to the solution;
applying the solution containing the colorant to the fibers; and
passing the fiber through a dryer to remove excess solvent.

16. The method of claim 15, further comprising applying an overcoat to the suture.

17. The method of claim 16, further comprising including a high molecular weight co-polymer in the overcoat.

18. The method of claim 16, further comprising a bioactive agent.

19. The method of claim 18, further comprising an anti-microbial, anti-thrombogenic, tissue healing/growth or cell attachment agent.

20. The method of claim 19, further comprising adding anti-microbial agent triclosan, and glyceryl monostearate.

21. A method of coating fibers used to create a medical device comprising:
creating a solution comprising:
a polymer;
a solvent;
adding a colorant to the solution;
applying the solution containing the colorant to the fibers;
passing the fiber through a dryer to remove excess solvent; and
adjusting the percentage of colorant in the solution based on suture size.

22. The method of claim 21, further comprising reducing the percentage of colorant in the solution for larger sutures.

23. The method of claim 21, further comprising applying an overcoat including a high molecular weight co-polymer.

24. The method of claim 21, further comprising a bioactive agent.

25. The method of claim 24, further comprising an anti-microbial, anti-thrombogenic, tissue healing/growth or cell attachment agent.

26. The method of claim 25, further comprising anti-microbial agent triclosan, and glyceryl monostearate.
